(19) United States
(12) Patent Application Publication
(10) Pub. No.: US 2021/0353136 A1
(43) Pub. Date: Nov. 18, 2021

(54) CORNEAL CONTACT TYPE OPHTHALMIC DIGITAL MICROSCOPE

(71) Applicant: MSC LAB Co., Ltd., Gwangju (KR)

(72) Inventors: Seon Ho Kim, Gwangju (KR); Sun Taek Lim, Gwangju (KR); Chang Ryong Kim, Gwangju (KR); Jun Sung Lee, Gwangju (KR)

(73) Assignee: MSC LAB Co., Ltd., Gwangju (KR)

(21) Appl. No.: 16/872,396

(22) Filed: May 12, 2020

(51) Int. Cl. A61B 3/00; A61B 3/13; A61B 3/14; A61F 9/009; A61B 3/10

(52) U.S. Cl. CPC A61B 3/0008; A61B 3/13; A61B 3/14; A61B 3/102; A61B 3/0016; A61F 9/009; A61B 3/0075

(57) ABSTRACT

The present invention relates to a conical contact type ophthalmic digital microscope, and more particularly, to a conical contact type ophthalmic digital microscope allowing observation of a magnified eyeball while being placed above a cornea of a patient. The corneal contact type ophthalmic digital microscope according to the present invention includes a housing, an objective lens part installed below the housing and configured to come in contact with a cornea of an eyeball, an image sensor part installed in the housing and configured to capture the eyeball visible through the objective lens part and generate an eyeball image, a position adjuster configured to change a position of the image sensor part, and a control part configured to control operations of the image sensor part and the position adjuster and output the eyeball image through a display provided outside the housing.

### BACKGROUND

### 1. Field of the Invention

The present invention relates to a conical contact type ophthalmic digital microscope, and more particularly, to a corneal contact type ophthalmic digital microscope allowing observation of a magnified eyeball while being placed above a cornea of a patient.

### 2. Discussion of Related Art

The eyeball is anatomically divided into two different parts, i.e., the anterior segment and the posterior segment.

The anterior segment refers to a part including the cornea, iris, and lens, and the posterior segment refers to a part including vitreous, retina and choroid

Diseases of the anterior segment include conical opacity, cataracts, and glaucoma, and diseases of the posterior segment include an epiretinal membrane, macular hole, retinal detachment, diabetic retinopathy, age-related macular degeneration, vitreous hemorrhage, and chorioretinitis.

In order to diagnose and treat such ophthalmic diseases, an ophthalmic microscope capable of precisely observing the eyeball is used. In recent years, an ophthalmic microscope has been used that outputs a shape of an affected area magnified by an objective lens so that a plurality of operators can monitor the surgical process.

As illustrated in FIGS. 1 and 2, a conventional ophthalmic microscope includes a light source (3) configured to emit light by power, an optical cable (4) configured to connect the light source (3) so that an affected area of a patient is irradiated with light, an objective lens (5) configured to magnify an eyeball of the patient, and an eyepiece (6) configured to allow visual inspection of the magnified eyeball.

Using the ophthalmic microscope, when light emitted from the light source (3) brightly illuminates the eyeball of the patient who is lying on an operating table and fixed in a specific position, the eyeball which is highly magnified by the objective lens (5) may be observed through the eyepiece (6).

The above-described conventional ophthalmic microscope has the following problems.

First, since an operator should maintain a posture while keeping the face pressed against the eyepiece of the microscope, movement of the operator is highly restricted.

Second, since the eyeball of the patient should be brightly illuminated by emitting strong visible light thereto, there are problems in that glare and eye tissue damage may be caused by a strong light source and time is taken to restore eyesight after surgery.

Third, since some of the light source emitted to the eyeball is reflected from the cornea, the quality of an image observed through the eyepiece is degraded.

Fourth, there is a problem in that water should be continuously sprayed on the eyeball in order to prevent drying of the cornea during examination or surgery.

Fifth, there is a problem in that the operating room should be kept dark by turning off the lighting of the operating room in order to clearly observe the appearance of the eyeball.

Sixth, combining the ophthalmic microscope with other ophthalmic equipment is difficult and complicated. For example, optical coherence tomography (OCT) is required to emit light of a specific wavelength to an eyeball and observe a cross section of the structure of the eyeball, but combining the OCT with the conventional ophthalmic microscope is difficult. There have been attempts to apply the OCT through the ophthalmic microscope, but there is a problem in that the objective lens of the ophthalmic microscope continuously moves to adjust a focal point and, every time the objective lens moves, the height needs to be corrected to perform the OCT.

Also, in the case of selective laser trabeculoplasty (SLT), which is a surgical method for reducing an intraocular pressure in glaucoma patients who need to undergo laser treatment, since the operator should hold a gonioscopic lens with one hand, place the gonioscopic lens in front of the eyeball of the patient, aim a laser at a trabecular meshwork, and irradiate the trabecular meshwork with the laser while looking at an angle in the eyeball using the ophthalmic microscope, there is a problem in that performing the procedure is difficult and inconvenient.

Seventh, the conventional ophthalmic microscope has disadvantages in that it is bulky, very complex in structure, and expensive.

### RELATED ART DOCUMENT

1. Korean Unexamined Patent Application Publication No. 10-2004-0022870

2. Korean Unexamined Patent Application Publication No. 10-2004-0105613

### SUMMARY OF THE INVENTION

The present invention is directed to providing a conical contact type ophthalmic digital microscope allowing observation of a magnified eyeball while an objective lens is brought in direct contact with a cornea of a patient, thereby addressing the above-described problems of the conventional microscope with which an eyeball is observed while an objective lens is spaced apart from the eyeball at a predetermined distance.

The present invention is also directed to providing a corneal contact type ophthalmic digital microscope allowing an objective lens to come in contact with a cornea so that an optical system of the microscope is made efficient and digitalizing the optical system so that the microscope is small-sized, lightweight, and thus able to be used while being placed on the cornea.

To achieve the above-described objectives, the present invention provides a corneal contact type ophthalmic digital microscope including a housing, an objective lens part installed below the housing and configured to come in contact with a cornea of an eyeball, an image sensor part installed in the housing and configured to capture the eyeball visible through the objective lens part and generate an eyeball image, a position adjuster configured to change a position of the image sensor part, and a control part configured to control operations of the image sensor part and the position adjuster and output the eyeball image to the outside, wherein the position adjuster includes a mounting member on which the image sensor part is mounted, a vertical movement part configured to vertically move the mounting member, and a tilting part configured to adjust a slope of the mounting member and adjust a direction of the image sensor part.

The objective lens part may include a contact lens having a contact surface, which is configured to come in contact with the cornea, concavely formed at a lower portion, a lighting module installed above the contact lens and configured to emit light toward the cornea, and an optical lens installed above the lighting module and configured to allow visual inspection of the eyeball in a magnified state.

The lighting module may include a light transmission panel disposed between the contact lens and the optical lens and configured to transmit light and a light source installed at the light transmission panel.

A reflection part may be formed on a side surface of the contact lens in order to reflect external light, which is incident on an inner portion of the housing from the outside, in a specific direction of the eyeball.

The corneal contact type ophthalmic digital microscope may further include a movable lens part installed between the objective lens part and the image sensor part and configured to be vertically movable.

The corneal contact type ophthalmic digital microscope may further include a beam splitter installed between the image sensor part and the objective lens part and configured to cause external light, which is emitted toward an inner portion of the housing from the outside, to be incident on the objective lens part.

The external light may be laser light for optical coherence tomography (OCT) or laser treatment.

The image sensor part may include a pair of left and right image-capturing elements horizontally spaced apart and configured to capture the eyeball at different angles to generate the eyeball image.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating a conventional ophthalmic microscope;

FIG. 2 is a view illustrating a state in which an eyeball is observed using the conventional ophthalmic microscope;

FIG. 3 is a perspective view of an ophthalmic digital microscope according to an embodiment of the present invention;

FIG. 4 is a cross-sectional view of the ophthalmic digital microscope of FIG. 3;

FIG. 5 is a block diagram illustrating a configuration of the ophthalmic digital microscope of FIG. 3;

FIG. 6 is a block diagram illustrating a configuration of an ophthalmic digital microscope according to another embodiment of the present invention;

FIG. 7 is a perspective view illustrating a support ring configured to fix the ophthalmic digital microscope according to the present invention to a cornea;

FIG. 8 is a cross-sectional view illustrating a state in which the ophthalmic digital microscope according to the present invention is placed on the cornea using the support ring of FIG. 7;

FIG. 9 is a plan view showing a lighting module applied in FIG. 1;

FIG. 10 is a plan view illustrating another state of the lighting module;

FIG. 11 is a cross-sectional view of an ophthalmic digital microscope according to yet another embodiment of the present invention;

FIG. 12 is a cross-sectional view of an ophthalmic digital microscope according to still another embodiment of the present invention;

FIG. 13 is a cross-sectional view of an ophthalmic digital microscope according to still another embodiment of the present invention;

FIG. 14 is a cross-sectional view of an ophthalmic digital microscope according to still another embodiment of the present invention;

FIGS. 15 and 16 are cross-sectional views of a main portion for showing usage states of the ophthalmic digital microscope of FIG. 14;

FIG. 17 is a cross-sectional view of a main portion of an ophthalmic digital microscope according to still another embodiment of the present invention;

FIG. 18 is a cross-sectional view illustrating a usage state of the ophthalmic digital microscope of FIG. 17;

FIG. 19 is a schematic diagram of a usage state of the ophthalmic digital microscope of FIG. 11;

FIG. 20 is a perspective view illustrating another example of the support ring configured to fix the ophthalmic digital microscope according to the present invention to a cornea; and

FIG. 21 is a cross-sectional view showing a state in which a close contact pad applied in FIG. 20 is attached to an eyeball.

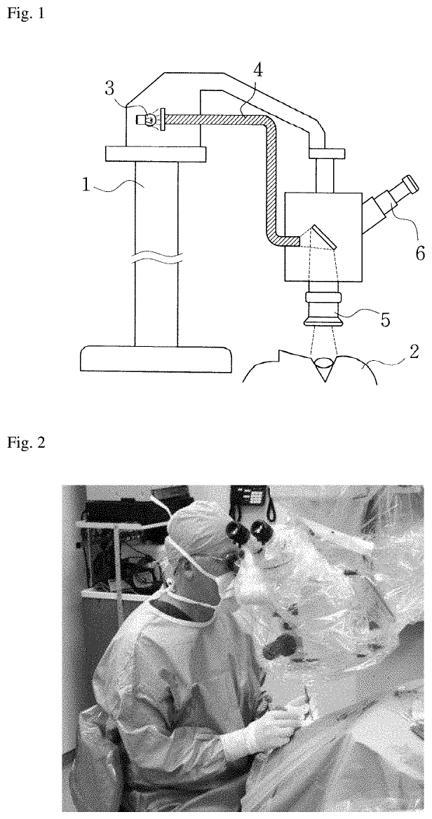

### DETAILED DESCRIPTION OF EXEMPLARY EMBODIMENTS

Hereinafter, a corneal contact type ophthalmic digital microscope will be described in detail with reference to the accompanying drawings.

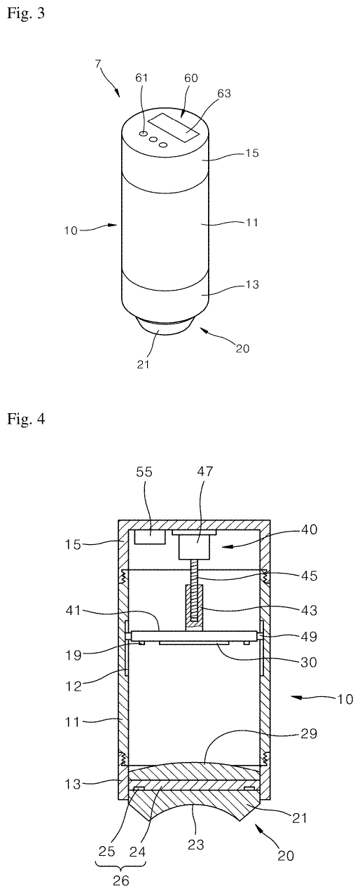

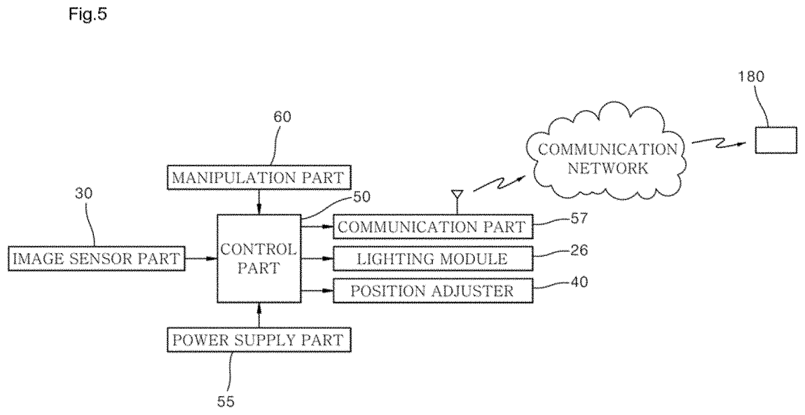

Referring to FIGS. 3 to 5, a corneal contact type ophthalmic digital microscope 7 according to an embodiment of the present invention includes a housing 10, an objective lens part 20 installed below the housing 10 and configured to come in contact with a cornea of an eyeball, an image sensor part 30 installed in the housing 10 and configured to capture the eyeball visible through the objective lens part 20 and generate an eyeball image, a position adjuster 40 configured to change a position of the image sensor part 30, and a control part 50 configured to control operations of the image sensor part 30 and the position adjuster 40.

The housing 10 may be formed of a hollow cylindrical structure. In the illustrated embodiment, the housing 10 has a structure divided into three parts. For example, the housing 10 may include a cylindrical upper case 11, a lower case 13 screw-coupled to a lower portion of the upper case 11, and a cap 15 screw-coupled to an upper portion of the upper case 11.

The upper case 11 and the lower case 13 have a vertically open structure, and the cap 15 has a structure with an open lower portion.

A lighting lamp 19 may be installed in the housing 10 to adjust the intensity of illumination. In the illustrated embodiment, the lighting lamp 19 is installed on a mounting member 41. A single lighting lamp 19 or two or more lighting lamps 19 may be installed. A light emitting diode (LED) capable of adjusting the intensity of illumination in stages is applied as the lighting lamp 19. The inner portion of the housing 10 may be kept bright or dark by the lighting lamp 19.

The objective lens part 20 is installed below the housing 10. When the housing 10 has the structure divided into three parts as illustrated, the objective lens part 20 may be installed at an inner side of the lower case 13.

In the present invention, the objective lens part 20 is different from that of the conventional ophthalmic microscope in that it has a structure coming in direct contact with a cornea of an eyeball. The objective lens part 20 allows visual inspection of the eyeball in a magnified state while being placed on the cornea of the eyeball.

The objective lens part 20 may include a contact lens 21 configured to come in contact with the cornea, a lighting module 26 installed above the contact lens 21 and configured to emit light toward the cornea, and an optical lens 29 installed above the lighting module 26 and configured to allow visual inspection of the eyeball in a magnified state.

The contact lens 21 is a lens that comes in contact with the cornea. The contact lens 21 is formed to have a flat upper portion. Also, a contact surface 23 configured to come in contact with the cornea is formed at a lower portion of the contact lens 21. The contact surface 23 is formed of a concavely curved surface. The contact surface 23 is formed as a curved surface corresponding to the shape of the cornea.

The contact lens 21 may be provided as a plurality of contact lenses 21 according to the radius of curvature and size of the contact surface 23, and an existing contact lens 21 may be replaced with another contact lens 21 suitable for a patient according to the shape of the cornea of the patient. In order to facilitate replacement of the contact lens 21, the contact lens 21 may be detachably coupled to the lower case 13. Also, the contact lens 21 may have a radius of curvature and a size that allow the contact lens 21 to come in contact with eyeballs of animals other than humans, in addition to coming in contact with eyeballs of humans.

The lighting module 26 is installed above the contact lens 21.

The lighting module 26 includes a light transmission panel 24 disposed between the contact lens 21 and the optical lens 29 and a light source 25 installed at the light transmission panel 24.

The light transmission panel 24 is formed in the shape of a disc whose upper portion and lower portion are flat. The lower portion of the light transmission panel 24 is pressed against the upper portion of the contact lens 21, and the upper portion of the light transmission panel 24 is pressed against the lower portion of the optical lens 29. The light transmission panel 24 is formed of transparent glass or synthetic resin capable of transmitting light.

The light source 25 is installed at a lower portion of the light transmission panel 24. An LED may be used as the light source 25. In order to install the light source 25 at the light transmission panel 24, a mounting groove may be formed in the lower portion of the light transmission panel 24.

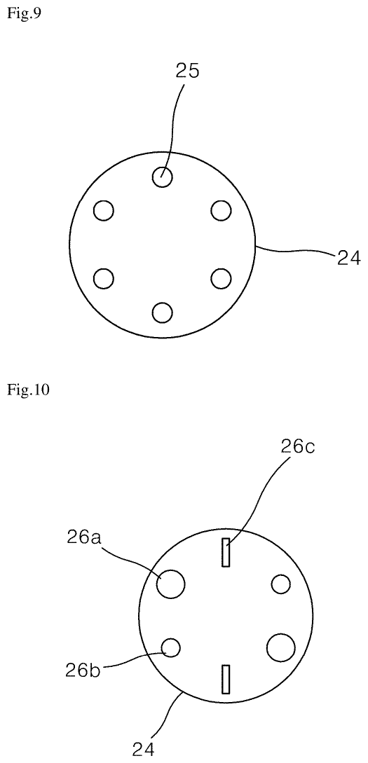

The LED used as the light source may be provided as a single LED or two or more LEDs. FIG. 9 illustrates a state in which, as the light source 25, a plurality of LEDs are disposed at the light transmission panel 24. The plurality of LEDs are disposed around an edge of the light transmission panel 24. Each LED may emit a circular beam toward the eyeball. The circular beam may be any one of parallel light, diffused light, and focused light.

Also, as illustrated in FIG. 10, as the light source, a plurality of LEDs may be disposed to selectively emit any one of a circular beam and a slit beam. Any one LED light source 26a emits a circular beam, another LED light source 26b emits a circular beam that is relatively narrower, and the remaining LED light source 26c emits a slit beam.

The slit beam is used when observing an optical cross-section of an eyeball. The slit beam is a long, thin ray of light and is generally used in a slit lamp microscope in order to observe an optical cross-section of an eyeball. Since the lighting module may generate a slit beam, the conical contact type ophthalmic digital microscope according to the present invention may also serve as a slit lamp microscope.

The optical lens 29 is installed above the lighting module 26 and allows visual inspection of the eyeball in a magnified state. An objective lens used in a general microscope may be applied as the optical lens 29. The optical lens 29 may be formed of a single lens or a combination of two or more lenses.

The image sensor part 30 is installed inside the housing 10, captures an eyeball visible through the objective lens part 20, and generates an eyeball image. In the illustrated embodiment, the image sensor part 30 is installed on the mounting member 41 of the position adjuster 40.

A high-resolution image-capturing element may be used as the image sensor part 30. A charge-coupled device (CCD), a complementary metal oxide semiconductor (CMOS), or the like that converts an image into an electrical signal and outputs the electrical signal may be used as the image-capturing element.

By the control part 50, the eyeball image captured by the image sensor part 30 may be output as an image on a display 180 provided outside the housing 10. For example, the display 180 may be a monitor installed in an examination room or an operating room.

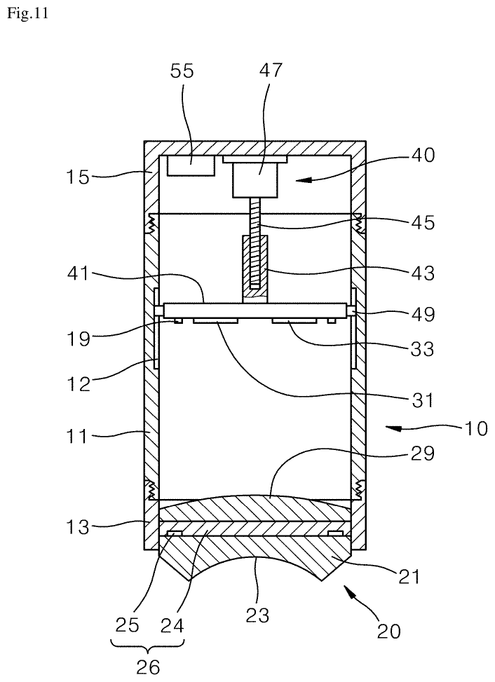

Meanwhile, in order to allow an examiner to view the eyeball image as a stereoscopic image, as illustrated in FIG. 11, the image sensor part may include a pair of left and right image-capturing elements 31 and 33. When the pair of left and right image-capturing elements are differentiated as a left-side image-capturing element 31 and a right-side image-capturing element 33 for convenience of description, the left-side image-capturing element 31 and the right-side image-capturing element 33 are horizontally spaced apart and capture a single eyeball at different angles.

In this case, the display separately outputs a left-side image, which is an eyeball image captured by the left-side image-capturing element 31, and a right-side image, which is an eyeball image captured by the right-side image-capturing element 33. As illustrated in FIG. 19, the display may be a virtual reality (VR) device 200. In this case, the VR device 200 may be configured so that short-range communication is possible with the corneal contact type ophthalmic digital microscope 7 according to the present invention. An examiner 205 may observe an eyeball of a patient through an image output from the VR device 200 while wearing the VR device 200 on the face. The image output from the VR device 200 may be separated into a left-side image and a right-side image. The eyeball image captured by the left-side image-capturing element 31 may be output as the left-side image from the VR device 200 through short-range communication, and the eyeball image captured by the right-side image-capturing element 33 may be output as the right-side image from the VR device 200 through short-range communication. Therefore, an examiner 1 may observe the eyeball image as a stereoscopic image through the VR device 200.

The position adjuster 40 changes a position of the image sensor part 30. The position adjuster 40 vertically moves the image sensor part 30 to adjust a focal point.

For example, the position adjuster 40 includes the mounting member 41 on which the image sensor part is mounted and a vertical movement part configured to vertically move the mounting member.

The mounting member 41 is formed in the shape of a plate. The image sensor part 30 is installed at the center of a lower portion of the mounting member 41.

The vertical movement part includes a protruding rod 43 coupled to the upper portion of the mounting member 41 and having a screw hole formed therein, a lead screw 45 screw-coupled to the protruding rod 43, a motor 47 configured to rotate the lead screw 45, and a guide protrusion 49 configured to guide vertical movement of the mounting member 41.

The protruding rod 43 is formed at the center of the upper portion of the mounting member 41. The protruding rod 43 is formed to be vertically long. A screw hole is formed in the protruding rod 43.

The lead screw 45 has screw threads formed on an outer circumferential surface and is screw-coupled to the screw hole of the protruding rod 43. The lead screw 45 is connected to the motor 47 and rotates.

The guide protrusion 49 is formed at each of left and right sides of the mounting member 41. The guide protrusion 49 is inserted into a guide groove 12 formed in an inner circumferential surface of an upper main body 11. The guide groove 12 is formed to be vertically long.

When the motor 47 installed at the cap 15 operates, the lead screw 45 rotates. Accordingly, the mounting member 41 moves vertically along a direction in which the lead screw 45 rotates.

The control part 50 controls operations of the image sensor part 30 and the position adjuster 40 to capture an eyeball image and vertically move the image sensor part 30. Also, the control part 50 outputs the eyeball image to an external display device. The eyeball image is implemented as an image through a display provided outside the housing 10.

The control part 50 may be installed at an inner portion of the cap 15 of the housing 10.

The control part 50 includes microprocesses and various driving circuits and controls operation of the corneal contact type ophthalmic digital microscope according to the present invention. Also, the control part 50 analyzes and processes an electrical signal input from the image sensor part 30.

Meanwhile, the corneal contact type ophthalmic digital microscope according to the present invention may include a power supply part 55 configured to provide power and a manipulation part 60 for manipulation.

A battery installed in the housing 10 may be used as the power supply part 55.

The manipulation part 60 may be provided at an upper portion of the cap 15. The manipulation part 60 may be provided as keys 61, including a power button, with which available functions may be set or provided as a touch panel. Also, a small display 63 may be provided at the upper portion of the cap 15 in order to visually show a manipulation state.

Also, the corneal contact type ophthalmic digital microscope according to the present invention may include a communication part 57 in order to output an eyeball image to the display 180 outside the housing 10.

The communication part 57 performs communication through a communication network. The communication part 57 may communicate via a wire or wirelessly. As a wireless communication network, a wireless local area network (WLAN) (Wi-Fi), wireless broadband (WiBro), world interoperability for microwave access (WiMAX), high-speed downlink packet access (HSDPA), HSUPA, LET, and the like may be used. As a short-range communication network for short-range communication, Bluetooth, radio frequency identification (RFID), infrared data association (IrDA), ultra wideband (UWB), ZigBee, near-field communication (NFC), and the like may be used.

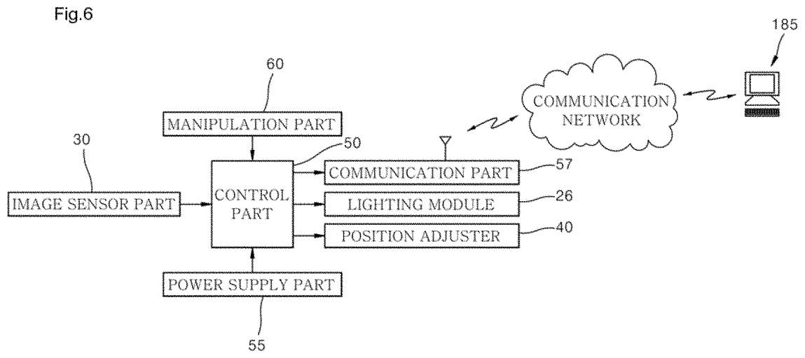

Also, as illustrated in FIG. 6, the corneal contact type ophthalmic digital microscope according to the present invention may further include an external terminal 185 configured to, through the control part 50, control operations of the image sensor part 30 and the position adjuster 40. In this case, the communication part 57 transmits various pieces of data to the external terminal 185, which is at a near distance or a far distance, or receives data and a control signal from the external terminal 185.

Examples of the external terminal 185 include a smartphone, a personal digital assistant (PDA), a desktop, a tablet PC, a notebook computer, and the like that may be applied to various wireless environments.

An eyeball image captured by the image sensor part 30 is output on a display of the external terminal 185. Also, the image sensor part 30 and the position adjuster 40 may be operated through the external terminal 185. The control part 50 may drive the image sensor part 30 and the position adjuster 40 according to a control signal of the external terminal 185.

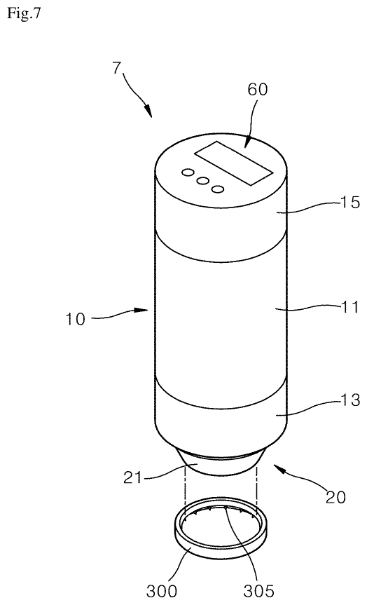

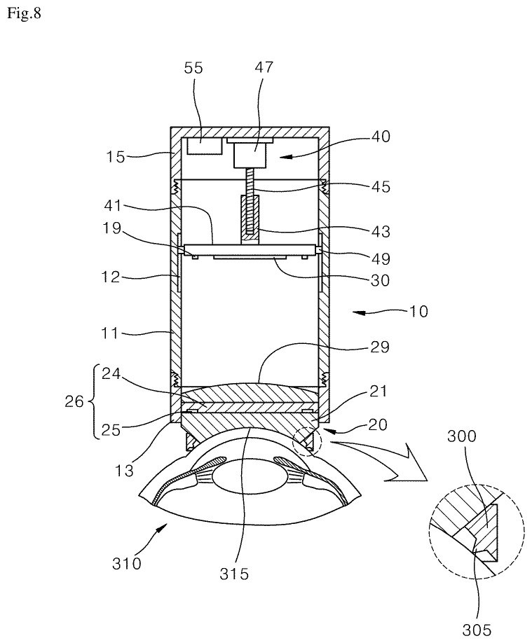

A method of use of the corneal contact type ophthalmic digital microscope according to the present invention will be briefly described with reference to FIGS. 7 and 8.

In order to use the corneal contact type ophthalmic digital microscope 7 according to the present invention, a support ring 300 configured to support the objective lens part 20 may be further included to maintain a state in which the objective lens part 20 is in contact with a cornea 315 of an eyeball 310.

The support ring 300 is formed in an annular shape. A plurality of protrusions 305 are formed at an inner side of the support ring 300. The protrusions 305 serve to suppress movement of the support ring 300 on the eyeball. The protrusions 305 may be formed in a conical shape that gradually narrows toward an end portion. The plurality of protrusions 305 may be formed at predetermined intervals to protrude from the support ring 300 toward a surface of the eyeball 310.

When the support ring 300 is placed on the eyeball 310, as the end portion of the protrusion 305 slightly presses the eyeball 310, the support ring 300 may be fixed at a specific position without moving.

First, upper and lower eyelids of a patient who is lying down are opened using an eyelid speculum and fixed, and then the support ring 300 is placed on the eyeball 310 of the patient. Then, the corneal contact type ophthalmic digital microscope according to the present invention is gently put down so that the contact lens 21 of the objective lens part 20 is inserted into the support ring 300.

An inclined surface corresponding to an outer side surface of the contact lens 21 may be formed at an inner side of the support ring 300, and the support ring 300 may stably support the contact lens 21. While the contact lens 21 is placed on the support ring 300, the contact surface of the contact lens 21 comes in contact with the cornea 315 of the eyeball.

In this state, the examiner or operator may observe the eyeball in a magnified state through the display outside the housing 10. Also, a suction ring may be used in addition to the support ring illustrated as an auxiliary tool. The suction ring may have a groove, which is capable of forming a negative pressure, formed in a lower portion coming in contact with the cornea and be fixed to the surface of the eyeball.

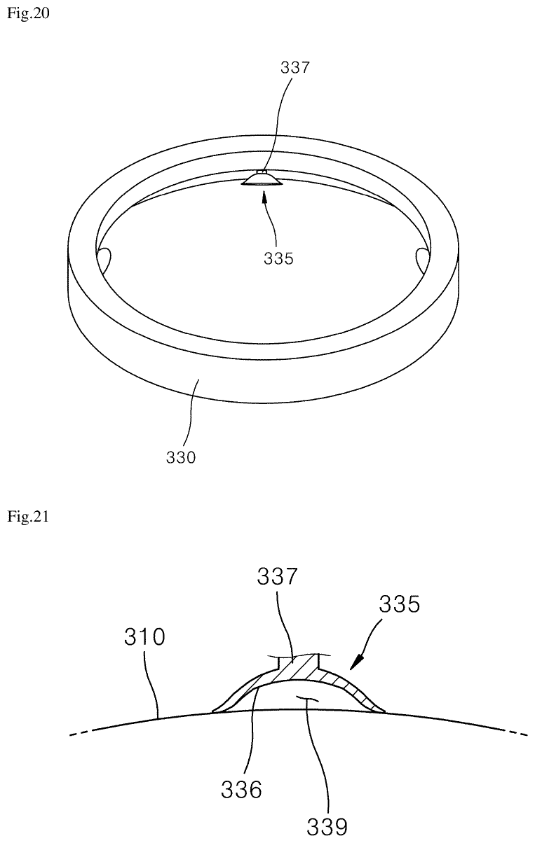

Also, a support ring illustrated in FIGS. 20 and 21 may be used. A support ring 330 illustrated in FIGS. 20 and 21 is different from the support ring 300 illustrated in FIGS. 7 and 8 in that a plurality of close contact pads 335 are installed instead of protrusions.

The plurality of close contact pads 335 are disposed at predetermined intervals. The close contact pads 335 are formed in the shape of a thin disc so as to come in surface contact with the eyeball 310. A front surface of the close contact pad 335 that faces the eyeball 310 constitutes a contact surface 336 that comes in contact with the eyeball 310. Also, a connection part 337 is formed at a rear surface of the close contact pad 335. The connection part 337 serves to connect the support ring 330 and the close contact pad 335.

The close contact pad 335 is formed in a shape that is depressed toward the center from an edge. Therefore, in the close contact pad 335, the contact surface 336 that comes in contact with the eyeball 310 is concavely formed. The contact surface 336 of the close contact pad 335 may be formed as a spherical surface or a curved surface that is non-spherical.

The close contact pad 335 may be deformed according to the degree to which it is pressed against the eyeball 310. The close contact pad 335 is formed of an elastically deformable material so that the close contact pad 335 may be restored to its original shape when separated from the eyeball 310. Examples of the elastically deformable material may include silicone that is harmless to the human body.

The above-described structure of the close contact pad 335 may maximize a contact area with the eyeball 310 which is formed of a non-spherical surface. By significantly increasing the contact area with the eyeball 310 and distributing a load, the close contact pad 335 may reduce a pressure applied to the eyeball 310 and effectively suppress movement of the support ring 330.

When the close contact pad 335 is brought into contact with the eyeball 310, as illustrated in FIG. 21, a hollow space 339 is formed between the eyeball 310 and the close contact pad 335 due to the concave structure of the close contact pad 335. In this state, when the close contact pad 335 is slowly pushed toward the eyeball 310 so that air is eliminated from a portion between the eyeball 310 and the close contact pad 335, a negative pressure is formed between the eyeball 310 and the close contact pad 335. The negative pressure formed in this case generates a force that causes the close contact pad 335 to be pressed against the eyeball 310. Therefore, the close contact pad 335 is attached to the surface of the eyeball 310 due to the negative pressure.

Therefore, when the support ring 330 is placed on the eyeball 310, the close contact pad 335 may be pressed against the eyeball 310, and the support ring 330 may be fixed at a specific position without moving.

As described above, according to the present invention, by digitalizing an optical system of the conventional ophthalmic microscope so that the microscope is small-sized and lightweight, the corneal contact type ophthalmic digital microscope according to the present invention may be used while the objective lens part is placed on the cornea using the support ring.

Accordingly, according to the present invention, because an eyeball may be magnified and observed while the objective lens part is brought in direct contact with a cornea of a patient, it is possible to address problems of the conventional ophthalmic microscope with which an eyeball is observed while an objective lens part is spaced apart from the eyeball at a predetermined distance.

For example, when the conventional ophthalmic microscope is used, because the examiner or operator should maintain his or her posture while keeping his or her face pressed against the eyepiece of the microscope, movement of the examiner or operator is highly restricted. On the other hand, according to the present invention, because the examiner or operator observes an image output through a display installed in the examination room or operating room, freedom of movement of the examiner or operator is very high.

Also, when the conventional ophthalmic microscope is used, because an eyeball of a patient should be brightly illuminated by emitting strong visible light thereto, glare and eye tissue damage may be caused by a strong light source. On the other hand, according to the present invention, because direct contact with an eyeball allows the eyeball to be observed with relatively weak light, glare and eye tissue damage may be minimized.

Also, when the conventional ophthalmic microscope is used, because some of the light source emitted to the eyeball is reflected from the cornea, the quality of an image observed through an eyepiece is degraded. On the other hand, according to the present invention, because the objective lens part comes in direct contact with the cornea, reflection of light from the cornea does not occur such that the quality of an image is improved, and a field of view may be designed to be wider as compared to the conventional ophthalmic microscope.

Also, when the conventional ophthalmic microscope is used, because an objective lens and an eyeball are spaced apart, there is a problem in that water should be continuously sprayed on the eyeball in order to prevent drying of the cornea during observation or surgery. On the other hand, according to the present invention, it is sufficient to have a viscous material, which serves as a lubricant, dropped onto the eyeball only at the beginning.

Also, when the conventional ophthalmic microscope is used, an examination room or an operating room should be kept dark by turning off the lighting in order to clearly observe the appearance of the eyeball. On the other hand, according to the present invention, because the inner portion of the housing may be kept dark, there is no need to keep the examination room or operating room dark.

Also, while the conventional ophthalmic microscope is bulky, very complex in structure, and expensive, the corneal contact type ophthalmic digital microscope according to the present invention is small-sized, lightweight, and very cheap to manufacture. Also, while the conventional ophthalmic microscope is not suitable for use on animals, the corneal contact type ophthalmic digital microscope according to the present invention is suitable for use on animals and thus may be used in treating cataracts in pets or wild animals and examining the retina of the pets or wild animals.

Meanwhile, according to another embodiment of the present invention, a movable lens part 70 may be further included.

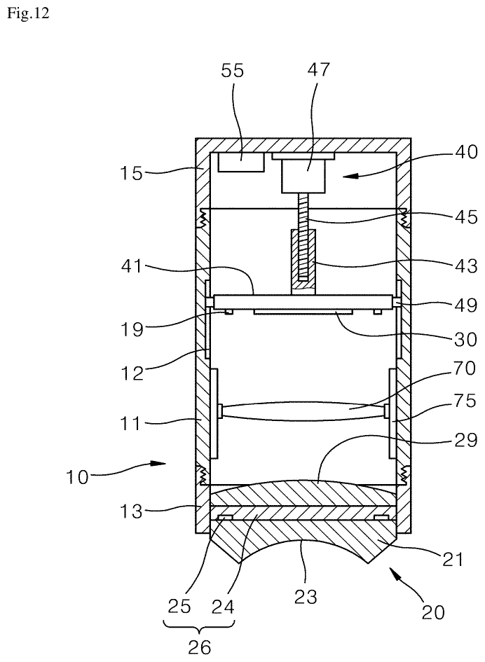

Referring to FIG. 12, the movable lens part 70 is installed between the objective lens part 20 and the image sensor part 30. The movable lens part 70 may be formed of a single lens or a combination of a plurality of lenses. The movable lens part 70 vertically moves and is used for zooming in and out and adjusting clarity of an image.

Various moving means may be used in order to vertically move the movable lens part 70. In the illustrated embodiment, a pair of linear motors 75 are used as the moving means. The linear motor is formed in a structure in which a mover is coupled to stators arranged in a straight line. When power is supplied, the mover of the linear motor 75 moves in a straight line. The linear motor 75 is advantageous in terms of size reduction and position control.

The linear motor 75 is installed at an inner side surface of the upper main body 11 of the housing 10. The movable lens part 70 is coupled to the mover of the linear motor 75. When the control part operates the linear motor 75, the movable lens part 70 may be vertically movable.

Meanwhile, the corneal contact type ophthalmic digital microscope according to the present invention may further include a beam splitter configured to cause external light, which is emitted toward the inner portion of the housing from the outside, to be incident on the objective lens part.

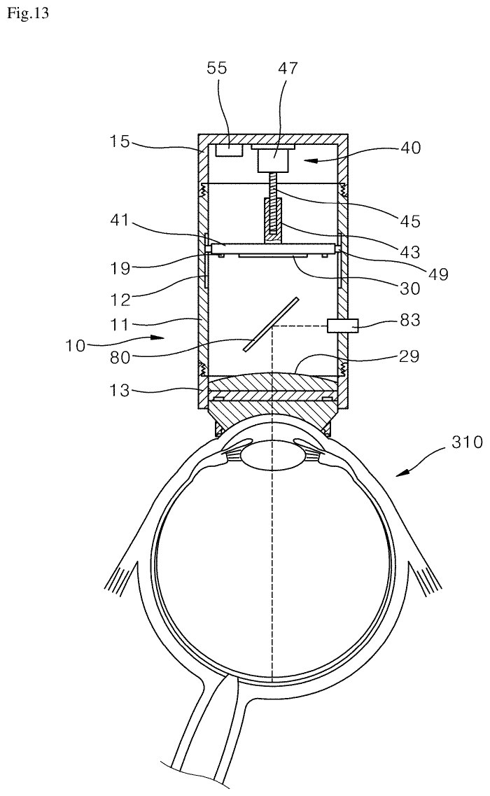

Referring to FIG. 13, a beam splitter 80 is installed between the image sensor part 30 and the objective lens part 20. The beam splitter 80, which is a kind of spectroscope, may be installed to be inclined, reflect external light emitted toward the inner portion of the housing 10 from the outside, and transmit the external light to the objective lens part 20.

For example, the external light may be laser light for OCT or laser treatment. Examples of the laser treatment may include laser treatment of peripheral retina and selective laser trabeculoplasty (SLT).

The external light is incident on the inner portion of the housing 10 from the outside through a probe 83 coupled to a side surface of the housing 10. The probe 83 is connected to an external device that outputs the external light. An insertion hole into which the probe 83 may be inserted may be formed in the side surface of the housing 10 so that the probe 83 may be coupled to the side surface of the housing 10. Also, alternatively, a window may be formed on the side surface of the housing 10, and a probe may be disposed outside the window.

The external light emitted toward the inner portion of the housing 10 is reflected by the beam splitter 80 and incident on the eyeball 310 by passing through the objective lens part 20.

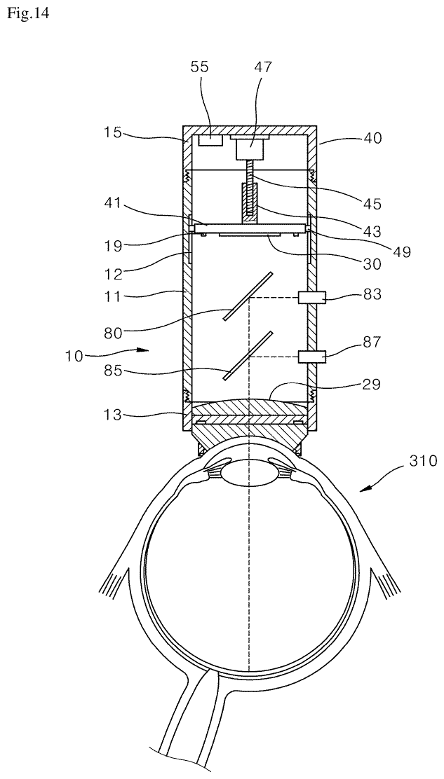

Also, when OCT and laser treatment are performed simultaneously, as illustrated in FIG. 14, two beam splitters 80 and 85 may be installed so that each laser light is incident on the eyeball.

The two beam splitters 80 and 85 are installed to be vertically spaced apart. Also, in order to cause external light to be incident on each of the beam splitters 80 and 85, two probes 83 and 87 are installed at the side surface of the housing 10. In this case, two insertion holes into which the two probes 83 and 87 may be inserted may be formed in the side surface of the housing 10 so that the two probes 83 and 87 may be coupled to the side surface of the housing 10.

Laser light for OCT is emitted to the inner portion of the housing 10 by the probe 83 disposed at an upper portion of the two probes 83 and 87, and laser light for laser treatment is emitted toward the inner portion of the housing 10 by the probe 87 disposed at a lower portion of the two probes 83 and 87. In this case, a cross-sectional image of an eyeball may be checked in real time while laser treatment is being performed.

External light may be transmitted to a specific position on an eyeball by adjusting an angle of a beam splitter or a probe. Although not illustrated, the angle of the beam splitter or probe may be adjusted using various known actuators.

Also, in order to further diversify a path of external light incident on an eyeball, a reflection part may be formed on a side surface of a contact lens.

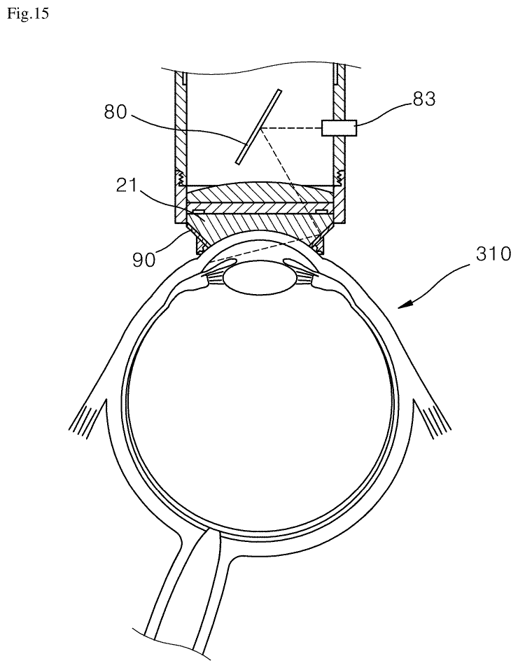

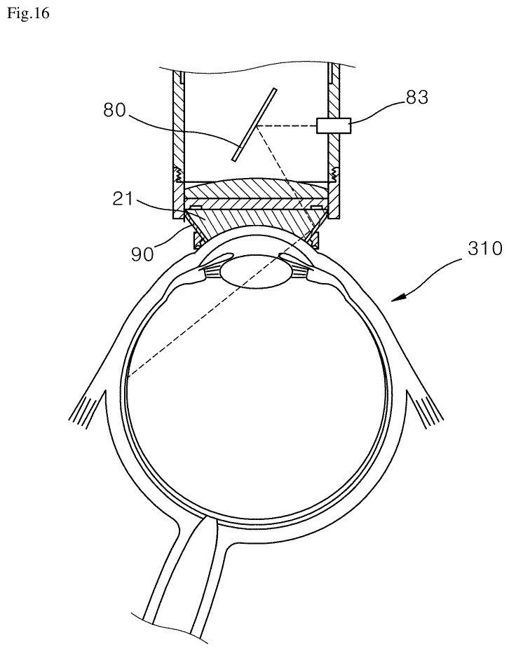

Referring to FIGS. 15 and 16, a reflection part 90 is formed on an inclined side surface of the contact lens 21. The reflection part 90 may be formed by attaching a surface of a mirror or the like to the side surface of the contact lens 21. Also, a reflective layer capable of reflecting light may be formed by being coated on the side surface of the contact lens 21.

The external light reflected from the beam splitter 80 is incident on the reflection part 90, and the external light incident on the reflection part 90 is reflected again and incident on a specific position on the eyeball 310. A direction in which the external light is reflected may be adjusted by adjusting an angle of the beam splitter 80 or the probe 83 or adjusted using contact lenses 21 whose side surfaces are inclined at different slopes. FIGS. 15 and 16 illustrate states in which a reflection part is formed at different angles using contact lenses whose side surfaces are inclined at different slopes. By making an angle of reflection small as illustrated in FIG. 15, a front angle may be examined, and thus the corneal contact type ophthalmic digital microscope according to the present invention may be utilized in a diagnostic examination for glaucoma, SLT, or the like. By making an angle of reflection large as illustrated in FIG. 16, the corneal contact type ophthalmic digital microscope according to the present invention may be utilized in examination or laser treatment of peripheral retina.

In this way, according to the present invention, a cross-sectional image of an eyeball may be checked in real time while laser treatment is being performed. This is particularly useful for trabeculoplasty using laser light. SLT is a method in which laser light is emitted toward a trabecular meshwork to destroy or contract the trabecular meshwork so that a pore size of the trabecular meshwork is enlarged and the amount of aqueous humor being drained is increased, thereby reducing an intraocular pressure. When the corneal contact type ophthalmic digital microscope according to the present invention is used, because observation is possible up to the episcleral vessel, whether a pore has sufficiently reached the Schlemm's canal may be checked during SLT surgery. Also, an effect of SLT may be enhanced by checking a portion where the Schlemm's canal is large and healthy. Also, Schlemm's canaloplasty, which is a new surgical method, is possible by finding a portion where the Schlemm's canal is not in good condition. In addition, ultimately, laser trabeculosclerostomy surgery may be attempted by finding a healthy episcleral vessel and forming a pore from the trabecular meshwork to the episclera so as to substitute for the existing trabeculoplasty surgery.

Also, in FIGS. 13 to 16, an OCT device may be connected to the upper portion of the housing, and the image sensor part may be disposed at the side surface of the housing.

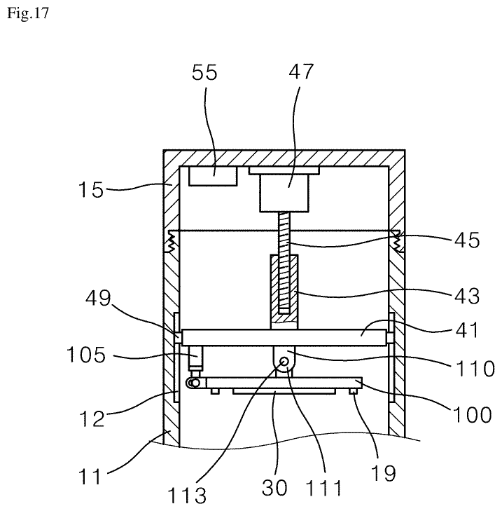

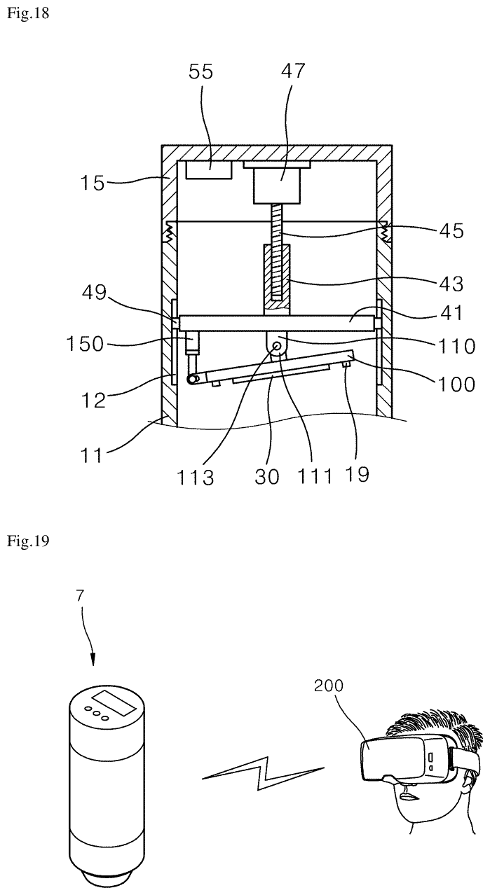

Meanwhile, FIGS. 17 and 18 illustrate another example of a position adjuster configured to change a position of the image sensor part 30. The illustrated position adjuster may not only vertically move the image sensor part 30 but also adjust a slope thereof.

Referring to FIGS. 17 and 18, the position adjuster includes a mounting member 100 on which the image sensor part 30 is mounted, a vertical movement part configured to vertically move the mounting member 100, and a tilting part configured to adjust a slope of the mounting member 100 to adjust a direction of the image sensor part 30.

The mounting member 100 is formed in the shape of a plate. The image sensor part 30 is installed at the center of a lower portion of the mounting member 100, and a lighting lamp 19 is installed at both sides of the image sensor part 30.

The vertical movement part includes a lifting/lowering plate 41, a protruding rod 43 coupled to an upper portion of the lifting/lowering plate 41 and having a screw hole formed therein, a lead screw 45 screw-coupled to the protruding rod 43, a motor 47 configured to rotate the lead screw 45, and a guide protrusion 49 configured to guide vertical movement of the lifting/lowering plate 41.

The lifting/lowering plate 41 is formed in the shape of a plate. The protruding rod 43 is formed at the center of an upper portion of the lifting/lowering plate 41. The protruding rod 43 is formed to be vertically long. A screw hole is formed in the protruding rod 43.

The lead screw 45 has screw threads formed on an outer circumferential surface and is screw-coupled to the screw hole of the protruding rod 43. The lead screw 45 is connected to the motor 47, which is installed at the cap 15, and rotates.

The guide protrusion 49 is formed at each of left and right sides of the lifting/lowering plate 41. The guide protrusion 49 is inserted into a guide groove 12 formed in an inner circumferential surface of an upper main body 11. The guide groove 12 is formed to be vertically long.

When the motor 47 operates, the lead screw 45 rotates. Accordingly, the lifting/lowering plate 41 vertically moves along a direction in which the lead screw 45 rotates.

The tilting part includes a hinge part configured to hinge-couple the lifting/lowering plate 41 and the mounting member 100 and an actuator 150 installed at the lifting/lowering plate 41 and configured to adjust the slope of the mounting member 100.

The hinge part includes a first bracket 110 formed at a lower portion of the lifting/lowering plate 41, a second bracket 111 formed at an upper portion of the mounting member 100, and a rotating shaft 113 configured to couple the first bracket 110 and the second bracket 111.

A cylinder or a solenoid value having a small size may be used as an actuator 105. The actuator 105 is hinge-coupled to one side of the mounting member 100. The mounting member 100 may be maintained in a horizontal state as illustrated in FIG. 17 or tilted as illustrated in FIG. 18 due to operation of the actuator 105. The operation of the actuator 105 is controlled by the control part.

By adjusting the slope of the mounting member 100 as described above, a direction of the image sensor part 30 may be adjusted to various directions. In this way, when external light is incident on a portion surrounding an eyeball instead of being incident on the center of the eyeball, the image sensor part 30 may be adjusted to correspond to a direction in which the external light is incident.

As described above, according to the present invention, by digitalizing an optical system of a microscope so that the microscope is small-sized and lightweight, the microscope can be used while being placed on the cornea.

Therefore, according to the present invention, because an eyeball can be magnified and observed while an objective lens is brought in direct contact with a cornea of a patient, it is possible to address various problems of the conventional microscope with which an eyeball is observed while an objective lens is spaced apart from the eyeball at a predetermined distance.

In addition, according to the present invention, because it is very easy to link the microscope with other ophthalmic equipment, the microscope can be used for OCT or various kinds of laser treatment.

The present invention has been described by referring to exemplary embodiments, but the above description is merely illustrative, and those of ordinary skill in the art should understand that various modifications and other equivalent embodiments are possible from the above embodiments. Therefore, the actual scope of the present invention should be defined only by the claims below.

What is claimed is:

1. A corneal contact type ophthalmic digital microscope comprising:
a housing;
an objective lens part installed below the housing and configured to come in contact with a cornea of an eyeball;
an image sensor part installed in the housing and configured to capture the eyeball visible through the objective lens part and generate an eyeball image;
a position adjuster configured to change a position of the image sensor part; and
a control part configured to control operations of the image sensor part and the position adjuster and output the eyeball image to the outside,
wherein the position adjuster includes a mounting member on which the image sensor part is mounted, a vertical movement part configured to vertically move the mounting member, and a tilting part configured to adjust a slope of the mounting member and adjust a direction of the image sensor part.

2. The corneal contact type ophthalmic digital microscope of claim 1, wherein the objective lens part includes a contact lens having a contact surface, which is configured to come in contact with the cornea, concavely formed at a lower portion, a lighting module installed above the contact lens and configured to emit light toward the cornea, and an optical lens installed above the lighting module and configured to allow visual inspection of the eyeball in a magnified state.

3. The corneal contact type ophthalmic digital microscope of claim 2, wherein the lighting module includes a light transmission panel disposed between the contact lens and the optical lens and configured to transmit light and a light source installed at the light transmission panel.

4. The corneal contact type ophthalmic digital microscope of claim 2, wherein a reflection part is formed on a side surface of the contact lens in order to reflect external light, which is incident on an inner portion of the housing from the outside, in a specific direction of the eyeball.

5. The corneal contact type ophthalmic digital microscope of claim 1, further comprising a movable lens part installed between the objective lens part and the image sensor part and configured to be vertically movable.

6. The corneal contact type ophthalmic digital microscope of claim 1, further comprising a beam splitter installed between the image sensor part and the objective lens part and configured to cause external light, which is emitted toward an inner portion of the housing from the outside, to be incident on the objective lens part.

7. The corneal contact type ophthalmic digital microscope of claim 6, wherein the external light is laser light for optical coherence tomography (OCT) or laser treatment.

8. The corneal contact type ophthalmic digital microscope of claim 1, wherein the image sensor part includes a pair of left and right image-capturing elements horizontally spaced apart and configured to capture the eyeball at different angles to generate the eyeball image.
